SEQUENCE LISTING (5 sequences)

SEQ ID NO: 1 - DNA, 1368 bp, synthetic construct
  source 1..1368: mol_type = other DNA
atgaccaccctgagcccgtctgaaggtaaagcgcagatccgtgcgctgctgaacctgattaacaccagcgcggaacaggcaatcgctgaatacgataaacaggaatgcgacattccgagcctgacctctggtgaaccgcacccgatggatgatcgtctgccgtccctggaactgaaaaacaccctgcgtattctggaaggtgcgtgcgcgcagctgtgcgttaccctggccccgccggcgcacaccatgctgaactatagcatggatgtgctggttccgtcttgcatcagcaccgttatccaggcgggtgttgcgccgctgctggcgaaacacccgaaaggcctgcacattgatgttctgagcaaagaaaccggcattcacccgcagaaactggcaaccattctgcgtctgctgatcctgaactactgcttccaggaagttgaatctaacgttttcgcgaacaaccgtctgagcctgaccctgctgccggaaaccagcgttgttgatattctggatctgaaaaccggtgaaatgcaccgtaaagcaaccctgtgggtttacgatgcgctggttgatccggacttcggtccgacctacgacggtaacaaatccccgctggtttacgcactgcgccgtgaaggtttcgatggttctctgtacgattacctacaaacccagccgggcgcggttgcacgtttcgctcgtgcaatgctgggtttcagcgtgagccgtggcctgatgaacctgctgaacgttttcccgtggcaggaactggcgccgggctctaccgtgtgcgacctgggcggtggtaacggtaacacctctatcgaaatcgcgaaaaaattcccgcacctgaaagttcacctccaggatctgccggataccatcgaagaagcgaaagtgttctggaaagaagaatacccggacgcgatcaaagatagccgcgttgcgttcaccccgatcgacttcttcaaacaggcgccggttccggaccaggacatctactacatcagccagattgttcataactggggtgatgaagattgcatcaccctgctgaaaaacattcgttctgcgatgagcccgaaaagccgtctgctgatcaacgattacctggcttctcacctggataaaacctctatcgcgaaccagcacccatctctgccgcgtgcgccgtacccgctgtctccgggcttcggtcgtggtatggcgcgtacctacaccggtgattacaccatgctggttgtttgcaacagccgtgaacgctctctggaagatttcatcgaactgtgcagcgcggcggatctgaaattcgttcgtgtttgggatctggctgaaaccagcgttaccgaatttgttccggcgtaa

SEQ ID NO: 2 - DNA, 498 bp, synthetic construct
  source 1..498: mol_type = other DNA
atgtcacaccccgcgttaacgcaactgcgtgcgctgcgctattgtaaagagatccctgccctggatccgcaactgctcgactggctgttgctggaggattccatgacaaaacgttttgaacagcagggaaaaacggtaagcgtgacgatgatccgcgaagggtttgtcgagcagaatgaaatccccgaagaactgccgctgctgccgaaagagtctcgttactggttacgtgaaattttgttatgtgccgatggtgaaccgtggcttgccggtcgtaccgtcgttcctgtgtcaacgttaagcgggccggagctggcgttacaaaaattgggtaaaacgccgttaggacgctatctgttcacatcatcgacattaacccgggactttattgagataggccgtgatgccgggctgtgggggcgacgttcccgcctgcgattaagcggtaaaccgctgttgctaacagaactgtttttaccggcgtcaccgttgtactaa

SEQ ID NO: 3 - DNA, 504 bp, synthetic construct
  source 1..504: mol_type = other DNA
atgcatgaaacaatatttactcaccaccccatcgactggctgaacgaagatgatgagtccgtgccgaacagcgtcctggattggctgcaagaacgtggtagcatgaccaagcgcttcgagcagcattgccagaaagttaccgttatcccgtacctggaacgctacattaccccagagatgctgtctgctgacgaggcggaacgccttccggagtcccagagatactggttgcgcgaagtgatcatgtatggtgataatattccgtggctgattggccgtaccctgatccctgaggaaaccttgacgaacgacgacaaaaaactggtggacatcggtcgtgttccgctgggtcgttacttgtttagccacgatagcctgacgcgtgattatattgacatcggcaccagcgcggaccgttgggttcgtcgtagcctgttacgcttgtcgcaaaagccgctgttgctcactgaaattttcctgccggagtctccggcatatcgttaa

SEQ ID NO: 4 - DNA, 1053 bp, synthetic construct
  source 1..1053: mol_type = other DNA
atgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcaatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaa

SEQ ID NO: 5 - DNA, 2379 bp, synthetic construct
  source 1..2379: mol_type = other DNA
atgtccaacaatggctcgtcaccgctggtgctttggtataaccaactcggcatgaatgatgtagacagggttgggggcaaaaatgcctccctgggtgaaatgattactaatctttccggaatgggtgtttccgttccgaatggtttcgccacaaccgccgacgcgtttaaccagtttctggaccaaagcggcgtaaaccagcgcatttatgaactgctggataaaacggatattgacgatgttactcagcttgcgaaagcgggcgcgcaaatccgccagtggattatcgacactcccttccagcctgagctggaaaacgccatccgcgaagcctatgcacagctttccgccgatgacgaaaacgcctcttttgcggtgcgctcctccgccaccgcagaagatatgccggacgcttcttttgccggtcagcaggaaaccttcctcaacgttcagggttttgacgccgttctcgtggcagtgaaacatgtatttgcttctctgtttaacgatcgcgccatctcttatcgtgtgcaccagggttacgatcaccgtggtgtggcgctctccgccggtgttcaacggatggtgcgctctgacctcgcatcatctggcgtgatgttctccattgataccgaatccggctttgaccaggtggtgtttatcacttccgcatggggccttggtgagatggtcgtgcagggtgcggttaacccggatgagttttacgtgcataaaccgacactggcggcgaatcgcccggctatcgtgcgccgcaccatggggtcgaaaaaaatccgcatggtttacgcgccgacccaggagcacggcaagcaggttaaaatcgaagacgtaccgcaggaacagcgtgacatcttctcgctgaccaacgaagaagtgcaggaactggcaaaacaggccgtacaaattgagaaacactacggtcgcccgatggatattgagtgggcgaaagatggccacaccggtaaactgttcattgtgcaggcgcgtccggaaaccgtgcgctcacgcggtcaggtcatggagcgttatacgctgcattcacagggtaagattatcgccgaaggccgtgctatcggtcatcgcatcggtgcgggtccggtgaaagtcatccatgacatcagcgaaatgaaccgcatcgaacctggcgacgtgctggttactgacatgaccgacccggactgggaaccgatcatgaagaaagcatctgccatcgtcaccaaccgtggcggtcgtacctgtcacgcggcgatcatcgctcgtgaactgggcattccggcggtagtgggctgtggagatgcaacagaacggatgaaagacggtgagaacgtcactgtttcttgtgccgaaggtgataccggttacgtctatgcggagttgctggaatttagcgtgaaaagctccagcgtagaaacgatgccggatctgccgttgaaagtgatgatgaacgtcggtaacccggaccgtgctttcgacttcgcctgcctaccgaacgaaggcgtgggccttgcgcgtctggaatttatcatcaaccgtatgattggcgtccacccacgcgcactgcttgagtttgacgatcaggaaccgcagttgcaaaacgaaatccgcgagatgatgaaaggttttgattctccgcgtgaattttacgttggtcgtctgactgaagggatcgcgacgctgggtgccgcgttttatccgaagcgcgtcattgtccgtctctctgattttaaatcgaacgaatatgccaacctggtcggtggtgagcgttacgagccagatgaagagaacccgatgctcggcttccgtggcgcgggccgctatgtttccgacagcttccgcgactgtttcgcgctggagtgtgaagcagtgaaacgtgtgcgcaacgacatgggactgaccaacgttgagatcatgatcccgttcgtgcgtaccgtagatcaggcgaaagcggtggttgaagaactggcgcgtcaggggctgaaacgtggcgagaacgggctgaaaatcatcatgatgtgtgaaatcccgtccaacgccttgctggccgagcagttcctcgaatatttcgacggcttctcaattggctcaaacgatatgacgcagctggcgctcggtctggaccgtgactccggcgtggtgtctgaattgttcgatgagcgcaacgatgcggtgaaagcactgctgtcgatggctatccgtgccgcgaagaaacagggcaaatatgtcgggatttgcggtcagggtccgtccgaccacgaagactttgccgcatggttgatggaagaggggatcgatagcctgtctctgaacccggacaccgtggtgcaaacctggttaagcctggctgaactgaagaaataa

(19) United States
(12) Patent Application Publication
(10) Pub. No.: US 2026/0201320 A1
(43) Pub. Date: Jul. 16, 2026

(54) RECOMBINANT ESCHERICHIA COLI FOR DE NOVO BIOSYNTHESIS OF ANISIC ACID AND PRODUCTION METHOD AND APPLICATION THEREOF

(71) Applicants: Xi'An Green Spring Technology Co., Ltd., Xi'an (CN); Northwest A&F University, Xianyang (CN)

(72) Inventors: Zi Wei Luo, Xianyang (CN); Renhan Li, Xianyang (CN); Qiang Hua, Xianyang (CN); Liying Liu, Xianyang (CN); Xiaozheng Li, Xianyang (CN); Shu-Wei Yuan, Xianyang (CN); Binbin Zhu, Xianyang (CN)

(21) Appl. No.: 19/412,942

(22) Filed: Dec. 9, 2025

(30) Foreign Application Priority Data
Jan. 14, 2025 (CN) 202510056056.7

(51) Int. Cl. C12N 1/20; C12N 9/10; C12N 9/12; C12N 9/88; C12P 7/40; C12R 1/19

(52) U.S. Cl. CPC C12N 1/20; C12N 9/1007; C12N 9/1085; C12N 9/1294; C12N 9/88; C12P 7/40; C12R 2001/19; C12Y 205/01054; C12Y 207/09002; C12Y 401/0304

(57) ABSTRACT

A recombinant Escherichia coli for de novo biosynthesis of p-anisic acid is provided, at least one of the following improvements has been made to a starting strain of E. coli: (1) overexpression of O-methyltransferase PSA-OMT1 from Pleurotus sapidus; overexpression of UbiC, a chorismic acid lyase derived from E. coli MG1655 or Providencia rustigianii; (2) based on (1), overexpression of a feedback inhibition mutant AroG^{D146N} derived from 3-deoxy-D-arabino-heptonate 7-phosphate, (DAHP) synthase of E. coli MG1655; (3) based on (2), overexpression of PpsA derived from phosphoenolpyruvate, (PEP) synthase, and knockout of pykF gene encoding a pyruvate kinase I. Therefore, the recombinant E. coli can achieve an accumulation of p-anisic acid in a fermentation broth during a fermentation process, and a maximum yield of p-anisic acid in shake flasks of recombinant E. coli can reach 284.9 mg/L, thereby laying a foundation for construction of high-yield p-anisic acid engineering bacteria.

### CROSS-REFERENCE TO RELATED APPLICATIONS

This application claims priority to Chinese Patent Application No. 202510056056.7, filed on Jan. 14, 2025, which is hereby incorporated by reference in its entirety.

### SEQUENCE LISTING

The present application contains a sequence listing which was filed electronically in XML format and is hereby incorporated by reference in its entirety. Besides, the XML copy is created on Dec. 9, 2025, is named “RECOMBINANT ESCHERICHIA COLI FOR DE NOVO SYNTHESIS OF ANISIC ACID AND PRODUCTION METHOD AND APPLICATION THEREOF” and is 11,204 bytes in sizes.

### TECHNICAL FIELD

The present disclosure relates to the field of genetic engineering and bioengineering technologies, and in particular, to a recombinant Escherichia coli for de novo biosynthesis of p-anisic acid and its production method and application.

### BACKGROUND

The chemical name of p-anisic acid is 4-methoxybenzoic acid, also known as p-anisic acid, para methoxybenzoic acid, etc. Its CAS registry number is 100-09-4. p-Anisic acid is mainly used as an intermediate in the synthesis of many drugs and spices, such as aneracetam, an anti-arrhythmic drug, and methyl anisate, which is used to modulate essence. Besides that, the p-anisic acid is often used as a flavoring and preservative agent in cosmetic formulations, as well as in food flavorings. At present, the p-anisic acid is mainly produced through chemical synthesis, and some are also produced through chemical and microbial transformations using trans anise and anise oil as raw materials. However, there have been no reports on the de novo biosynthesis of p-anisic acid using synthetic biology techniques. Chemical synthesis has problems such as high production cost, strict process conditions, many by-products, and easy pollution, and gradually be replaced by microbial fermentation method for de novo synthesis.

### SUMMARY

The main objective of the present disclosure is to provide a recombinant Escherichia coli for de novo biosynthesis of p-anisic acid, as well as a method and application for producing the p-anisic acid so as to at least solve problems of high cost and many by-products of chemical synthesis of p-anisic acid in the prior art.

In order to achieve the above objectives, the present disclosure provides a recombinant Escherichia coli strain for de novo biosynthesis of p-anisic acid, where at least one of the following improvements has been made to a starting strain of E. coli:
(1) overexpression of O-methyltransferase PSA-OMT1 from Pleurotus sapidus; overexpression of UbiC, a chorismic acid lyase derived from E. coli MG1655 or Providencia rustigianii;
(2) on the basis of (1), overexpression of a feedback inhibition mutant AroG^{D146N} derived from 3-deoxy-D-arabino-heptulosonate 7-phosphate, (DAHP) synthase of E. coli MG1655;
(3) on the basis of (2), overexpression of PpsA derived from phosphoenolpyruvate, (PEP) synthase, and knockout of pykF gene encoding a pyruvate kinase I;
a nucleotide sequence of codon optimized O-methyltransferase PSA-OMT1 is shown in SEQ ID No.: 1;
a nucleotide sequence of the chorismic acid lyase UbiC derived from E. coli MG1655 is shown in SEQ ID No.: 2;
a nucleotide sequence of codon optimized chorismic acid lyase UbiC derived from Providencia rustigianii is shown in SEQ ID No.: 3;
a nucleotide sequence of the feedback inhibition mutant AroG^{D146N} of DAHP synthase is shown in SEQ ID No.: 4;
a nucleotide sequence of the PEP synthase PpsA is shown in SEQ ID No.: 5.

In some embodiments of the present disclosure, the O-methyltransferase PSA-OMT1 and the chorismic acid lyase UbiC are expressed using pTrc99A as an expression vector.

In some embodiments of the present disclosure, the feedback inhibition mutant AroG^{D146N} of (DAHP synthase is expressed using pTrc99A or pBBR1Tac as an expression vector.

In some embodiments of the present disclosure, the PEP synthase PpsA is expressed using pCDFTac as an expression vector.

In some embodiments of the present disclosure, the recombinant E. coli is derived from E. coli MG1655 as a starting strain.

The present disclosure further provides a method for producing p-anisic acid, which includes using recombinant E. coli as a fermentation strain, and glucose is fermented as a substrate to produce the p-anisic acid.

In some embodiments of the present disclosure, the method includes: the recombinant E. coli is inoculated into a fermentation medium and cultured at 28-32° C. and 100-500 rpm for 48-72 hours; when OD600=0.5-0.8, isopropyl-beta-D-thiogalactopyranoside, (IPTG) with a final concentration of 0.1-1.0 mM is added for induction, after fermentation, a supernatant is separated and purified to obtain the p-anisic acid.

In some embodiments of the present disclosure, the fermentation medium includes glucose, MgSO₄·7H₂O, KH₂PO₄, (NH₄)₂HPO₄, Citric acid, L-methionine, trace metal salt solution; and deionized water, and a pH value of the fermentation medium is 6.5~7.5.

The present disclosure further provides an application of the method for producing p-anisic acid in the preparation of products containing p-anisic acid by microbial fermentation.

A recombinant Escherichia coli strain for de novo biosynthesis of p-anisic acid according to the technical solution of the present disclosure has been improved with at least one of the following modifications to made to a starting strain of E. coli: (1) overexpression of O-methyltransferase PSA-OMT1 from Pleurotus sapidus; overexpression of UbiC, a chorismic acid lyase derived from E. coli MG1655 or Providencia rustigianii; (2) on the basis of (1), overexpression of a feedback inhibition mutant AroG^{D146N} derived from DAHP synthase of E. coli MG1655; (3) on the basis of (2), overexpression of PpsA derived from PEP synthase, and knockout of pykF gene encoding the pyruvate kinase I; the nucleotide sequence of codon optimized O-methyltransferase PSA-OMT1 is shown in SEQ ID No.: 1; the nucleotide sequence of the chorismic acid lyase UbiC derived from E. coli MG1655 is shown in SEQ ID No.: 2; the nucleotide sequence of codon optimized chorismic acid lyase UbiC derived from Providencia rustigianii is shown in SEQ ID No.: 3; the nucleotide sequence of the feedback inhibition mutant AroG^{D146N} of DAHP synthase is shown in SEQ ID No.: 4; the nucleotide sequence of the PEP synthase PpsA is shown in SEQ ID No.: 5. Therefore, the recombinant E. coli in the present disclosure can achieve an accumulation of p-anisic acid in a fermentation broth during a fermentation process, and a maximum yield of p-anisic acid in shake flasks of recombinant E. coli can reach 284.9 mg/L, laying a foundation for the construction of high-yield p-anisic acid engineering bacteria in the future.

### BRIEF DESCRIPTION OF DRAWINGS

The accompanying drawings, which form a part of this application, are used to provide further understanding of the present disclosure. The illustrative embodiments and their descriptions of the present disclosure are used to explain the present disclosure and do not constitute undue limitation of the present disclosure.

FIG. 1 is a schematic diagram of an optional constructed biosynthesis pathway and genetic engineering modification of p-anisic acid in E. coli according to an embodiment of the present disclosure.

FIG. 2 shows an optional plasmid pLPAA01 profile according to an embodiment of the present disclosure.

FIG. 3 shows an optional plasmid pLPAA02 profile according to an embodiment of the present disclosure.

FIG. 4 shows an optional plasmid pLPAA03 profile according to an embodiment of the present disclosure.

FIG. 5 shows an optional plasmid pLPAA04 profile according to an embodiment of the present disclosure.

FIG. 6 shows an optional plasmid pLPAA05 profile according to an embodiment of the present disclosure.

FIG. 7 shows an optional plasmid pLPAA06 profile according to an embodiment of the present disclosure.

FIG. 8 shows a fermentation production of p-anisic acid by optional strains LPAA01~LPAA05 according to an embodiment of the present disclosure, along with corresponding plasmid diagrams.

FIG. 9 is a high performance liquid chromatography peak diagram of an optional standard of p-hydroxybenzoic acid and p-anisic acid, as well as an LPAA03 fermentation sample, according to an embodiment of the present disclosure.

FIG. 10 shows a fermentation production of p-anisic acid by optional strains LPAA05 and LPAA06 under different conditions according to an embodiment of the present disclosure.

### DESCRIPTION OF EMBODIMENTS

It should be noted that the embodiments and features in the embodiments of the present application can be combined with each other without conflict. The present disclosure will be described in detail below with reference to the accompanying drawings and in combination with embodiments.

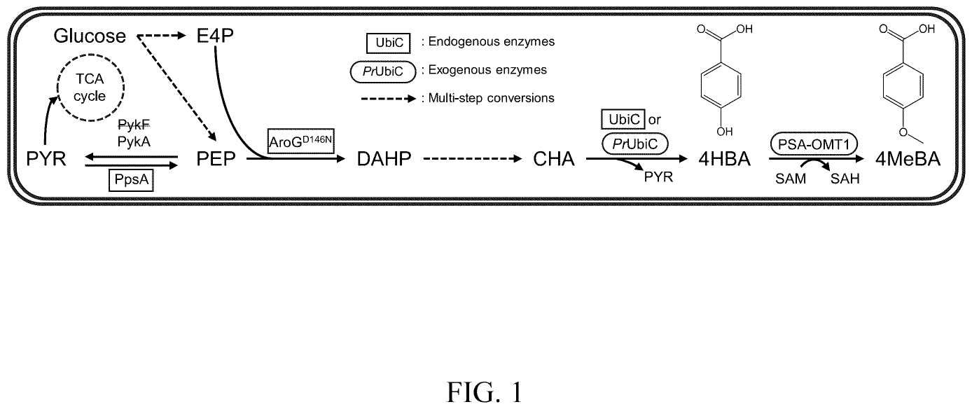

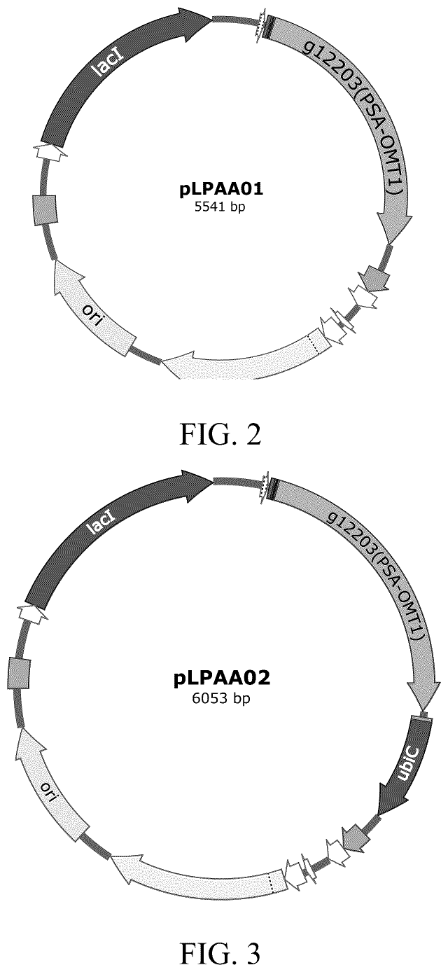

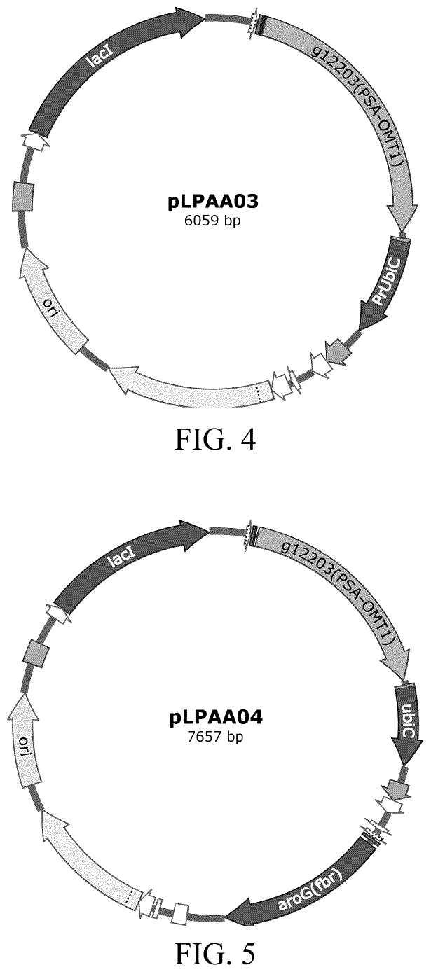

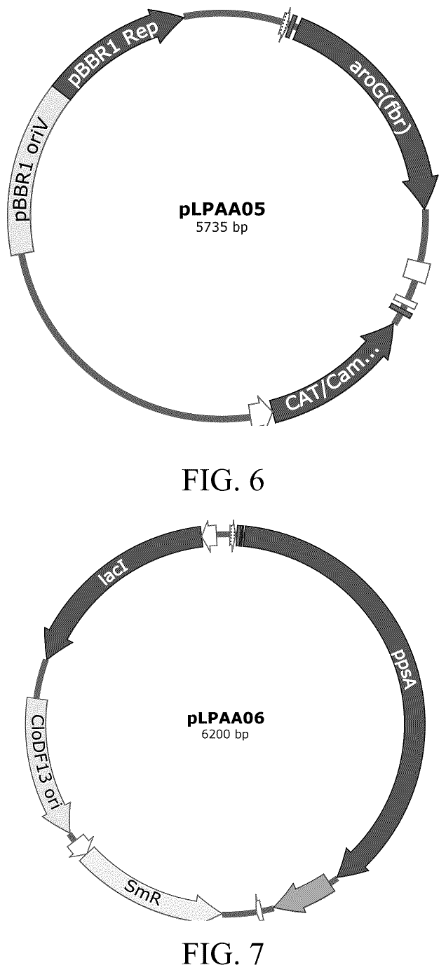

As shown in FIG. 1, glucose is first converted into PEP and erythrin-4-phosphate (E4P) through glycolysis and pentose phosphate pathways in E. coli cells. Most of these PEPs are catalyzed by pyruvate kinase I (PykF) and pyruvate kinase II (PykA) to convert into pyruvate (PYR), which then enters a tricarboxylic acid cycle (TCA cycle). PYR can be catalyzed to PEP by phosphoenolpyruvate synthase (PpsA). Therefore, by knocking out the pykF gene and overexpressing the ppsA gene, the synthesis of intracellular PEP can be enhanced. PEP and E4P are catalyzed by the DAHP synthase AroG to generate DAHP, which is then catalyzed by six enzymes via a Shikimate pathway to form chorismic acids (CHA). The mutant AroG^{D146N} of AroG can relieve a feedback inhibition effect of L-phenylalanine on its production and increase a carbon flow towards chorismic acids. UbiC, a chorismic acid lyase derived from E. coli or Providencia rustigianii, can cleave the chorismic acids into PYR and 4-hydroxybenzoic acid (4HBA). The g12203 gene derived from Pleurotus sapidus encodes an O-methyltransferase PSA-OMT1, which can use S-adenosyl-L-methionine (SAM) as a methyl donor to convert p-hydroxybenzoic acid to the p-anisic acid and generate S-adenosyl-L-homocysteine (SAH). Therefore, constructing this synthetic pathway in E. coli and enhancing the carbon flow towards this pathway is a good way to build a microbial production strain for the p-anisic acid, which has good application prospects.

In an implementation mode, this application has made at least one improvement to E. coli MG1655 as follows.

(1) Using pTrc99A plasmid as an expression vector to express the O-methyltransferase PSA-OMT1 from Pleurotus sapidus, and using pTrc99A plasmid as an expression vector to express the chorismic acid lyase UbiC from E. coli MG1655 or Providencia rustigianii, a recombinant E. coli strain capable of de novo synthesis of p-anisic acid from glucose as a substrate was preliminarily constructed. A nucleotide sequence (g12203^{opt}) of the O-methyltransferase PSA-OMT1 after codon optimization is shown in SEQ ID No.: 1. A nucleotide sequence of UbiC, an endogenous chorismic acid lyase in E. coli MG1655, is shown in SEQ ID No.: 2. A nucleotide sequence of the codon optimized chorismic acid lyase UbiC derived from Providencia rustigianii is shown in SEQ ID No.: 3.

(2) On the basis of (1), the pBBR1Tac plasmid was used as an expression vector to express a feedback inhibition mutant AroG^{D146N} of DAHP synthase derived from E. coli MG1655 so as to enhance the carbon flow of a Shikimate pathway and increase the concentration of precursor chorismic acids. A nucleotide sequence of the feedback inhibition mutant AroG^{D146N} of DAHP synthase is shown in SEQ ID No.: 4.

(3) On the basis of (2), the pCDFTac plasmid was used as an expression vector to express the PEP synthase PpsA, and a pykF gene encoding a pyruvate kinase I was knocked out to increase the concentration of the precursor PEP of DAHP. A nucleotide sequence of the PEP synthase PpsA is shown in SEQ ID No.: 5.

The present application further provides a method for producing p-anisic acid, including the recombinant E. coli constructed in the present application as a fermentation strain, and glucose as a substrate is fermented to produce the p-anisic acid.

After obtaining the recombinant E. coli, each recombinant E. coli strain was used as a fermentation strain, and glucose was used as the substrate to ferment and produce the p-anisic acid. A specific process is as follows: the p-anisic acid producing bacteria was inoculated into a fermentation medium, and conducting fermentation and cultivation at 28-32° C. and 100-500 rpm for 48-72 hours. After fermentation, a supernatant is taken and separated and it is purified to obtain the p-anisic acid. The fermentation medium contains glucose, MgSO₄·7H₂O, KH₂PO₄, (NH₄)₂HPO₄, Citric acid, L-methionine, trace metal salt solution; pH 6.5~7.5, the solvent is deionized water. Composition of trace metal salt solution: 10 g/L FeSO₄·7H₂O, 2.65 g/L CaCl₂·2H₂O, 2.2 g/L ZnSO₄·7H₂O, 0.58 g/L MnSO₄·5H₂O, 1 g/L CuSO₄·5H₂O, 0.1 g/L (NH₄)₆Mo₇O₂₄·4H₂O, 0.02 g/L Na₂B₄O₇·10H₂O, 10 mL/L 35% HCl, the solvent is deionized water.

Before fermentation of the recombinant E. coli strain, it was first inoculated into a LB medium and incubated overnight on a shaker at 37° C. and 200 rpm. Then, it was inoculated into the fermentation medium at a volume concentration of 1-5%. A total fermentation time is 60 hours. Where the fermentation involves inoculating the recombinant E. coli into the fermentation medium and culturing it until OD600=0.5~0.8, followed by induction with IPTG at a final concentration of 0.1~1.0 mM.

The method for producing p-anisic acid in this application can be applied to the preparation of products containing p-anisic acid through microbial fermentation.

Further illustrate the present disclosure through the following examples.

In the following examples, a final concentration of ampicillin (Ap) used is 100 mg/L, a final concentration of chloramphenicol (Cm) used is 34 mg/L, and a final concentration of streptomycin (Sm) used is 50 mg/L. The competent cells of E. coli used in the examples were E. coli DH5 α, a restriction endonuclease was purchased from TAKARA company, and a seamless cloning kit used in the recombination reaction was purchased from ABclonal Biotechnology Co., Ltd., but not limited to these companies.

### Example 1: Construction of pLPAA01~pLPAA03 Plasmids

The pTrc99A plasmid was subjected to single enzyme digestion using a restriction endonuclease NcoI to obtain a linearized plasmid vector fragment. Using PSA-OMT1_F and PSA-OMT1_R as primers, the g12203^{opt} gene encoding the fully synthesized O-methyltransferase PSA-OMT1 was amplified using a plasmid template containing the g12203^{opt} gene (sequence shown in SEQ ID No.: 1). A linear pTrc99A fragment after digestion and a g12203^{opt} gene fragment after amplification were subjected to agarose gel electrophoresis. Correct bands were cut and purified with gel recovery kit. Then, the seamless cloning kit was used for recombination reaction. After a reaction product was transformed into competent cells of E. coli, it was coated on the LB plate containing ampicillin sodium. Using Seq-pTrc99A_F and Seq-pTrc99A_R as primers, colony PCR was used to validate positive clones. Positive clones were sent to a sequencing company for sequencing to determine the successfully ligated plasmid pLPAA01.

Perform single enzyme digestion of pLPAA01 plasmid using a restriction endonuclease XbaI to obtain a linearized plasmid vector fragment. Simultaneously using ubiC_F and ubiC_R as primers and the E. coli genome as a template, the ubiC gene (sequence as shown in SEQ ID No.: 2) is amplified. Using PrubiC_F and PrubiC_R as primers and a plasmid containing the fully synthesized PrubiC^{opt} gene as a template, the PrubiC^{opt} gene (sequence shown in SEQ ID No.: 3) is amplified. The linear pLPAA01 fragment after digestion, the ubiC gene fragment after amplification, and the PrubiC^{opt} gene fragment after amplification were subjected to agarose gel electrophoresis, the correct bands were cut, and purified with gel recovery kit. The ubiC gene fragment was recombined with the purified pLPAA01 fragment using the seamless cloning kit. The reaction product was transformed into competent E. coli cells and coated onto the LB plates containing ampicillin sodium. Using Seq-pTrc99A_F and Seq-pTrc99A_R as primers, colony PCR was used to validate positive clones. Positive clones were sent to a sequencing company for sequencing to determine the successfully ligated plasmid pLPAA02. The PrubiC^{opt} gene fragment was recombined with the purified pLPAA01 fragment using the seamless cloning kit. The reaction product was transformed into competent E. coli cells and coated onto the LB plates containing ampicillin sodium. Using Seq-pTrc99A_F and Seq-pTrc99A_R as primers, colony PCR was used to validate positive clones. Positive clones were sent to a sequencing company for sequencing to determine the successfully ligated plasmid pLPAA03.

### Example 2: Construction of pLPAA04~pLPAA06 Plasmids

Using the pBBR1Tac plasmid as a template and pBBR1Tac_F and pBBR1Tac_R as primers, linearized plasmid fragments were amplified by PCR. And the aroGD^{146N} (sequence as shown in SEQ ID No.: 4) fragments aroG-F1 and aroG-F2 were amplified using the E. coli genome as a template, with aroG1_F and aroGf1_R, aroGf2_F and aroG2_R as primers, respectively. The linearized pBBR1Tac fragment, aroG-F1 fragment and aroG-F2 fragment were subjected to agarose gel electrophoresis, the correct bands were cut, and purified with gel recovery kit, and then the seamless cloning kit was used for recombination reaction. After the reaction product was transformed into competent cells of E. coli, it was coated on the LB plate containing chloramphenicol. Using Seq-pBBR1Tac-F and Seq-pTrc99A_R as primers, colony PCR was used to validate the positive clones. Positive clones were sent to a sequencing company for sequencing to determine the successfully connected plasmid pLPAA05.

Perform double enzyme digestion of pLPAA02 plasmid using restriction endonucleases KpnI and HindIII to obtain linearized plasmid vector fragments. Using ubiCT_F and ubiCT_R as primers and pLPAA02 plasmid as a template, amplify the ubiC gene and rrnB terminator. Using tac-aroG_F and tac-aroG_R as primers and pLPAA05 plasmid as a template, the tac-aroG^{D146N} fragment is amplified. The digested linear pLPAA02 fragment, the amplified ubiC gene, rrnB terminator fragment, and tac-aroG^{D146N} fragment were subjected to agarose gel electrophoresis. The correct bands were cut and purified with gel recovery kit. Then, the seamless cloning kit was used for recombination reaction. After the reaction product was transformed into competent cells of E. coli, it was coated on the LB plate containing ampicillin sodium. Using Seq-pTrc99A_F and Seq-pTrc99A_R as primers, colony PCR was used to validate positive clones. Positive clones were sent to a sequencing company for sequencing to determine the successfully ligated plasmid pLPAA04.

Performing double enzyme digestion of pCDFTac plasmid using restriction endonucleases EcoR I and Sac I to obtain linearized plasmid vector fragments. Simultaneously, using ppsA_F and ppsA_R as primers and the E. coli genome as a template, the ppsA gene (sequence as shown in SEQ ID No.: 5) is amplified. The linear pCDFTac fragment after digestion and the ppsA gene fragment after amplification were subjected to agarose gel electrophoresis. The correct bands were cut and purified with gel recovery kit. Then, the seamless cloning kit was used for recombination reaction. After the reaction product was transformed into competent E. coli cells, it was coated on the LB plate containing streptomycin sulfate. Using Seq-pCDFTac_F and Seq-pTrc99A_R as primers, colony PCR was used to validate the positive clones. Positive clones were sent to a sequencing company for sequencing to determine the successfully ligated plasmid pLPAA06.

### Example 3: Knockout of pykF Gene in E. coli MG1655

1) Preparation of Linearized Fragment KO-Δ pykF

Using the E. coli genome as a template, primers were used to amplify the target gene upstream and downstream sequences of 500 bp each as homologous arms using Δ pykF-up_F and Δ pykF-up_R, Δ pykF-down_F, and Δ pykF-down_R. DNA fragments Δ pykF-up and Δ pykF-down were obtained. Using PUTrc plasmid as a template and KO_F and KO_R as primers, the lox71-CmR-lox66 gene fragment was amplified, denoted as fKO. Using Δ pykF-up and fKO as templates and Δ pykF-up_F and KO_R as primers, overlapping extension PCR was performed to amplify the fragment up-fKO. Using up-fKO and Δ pykF-down as templates, and Δ pykF-up_F and Δ pykF-down_R as primers, overlapping extension PCR was performed to amplify the fragment KO-Δ pykF.

(2) Introduction of Auxiliary Plasmid pKD46

Preparation of E. coli MG1655 by electroporation: laboratory preserved E. coli MG1655 is inoculated into a test tube containing 5 mL LB, at 37° C., 200 rpm, and shaking for 12-16 hours. Inoculating 2% volume into 100 mL fresh LB medium, and adding L-arabinose with a final concentration of 10 mM, at 37° C., 200 rpm, and shaking for 1.5 hours, OD600=0.4-0.6. Centrifuging and discarding the supernatant, washing twice with 10% glycerol, and finally adding a certain amount of 10% glycerol to make a final volume to be 600-800 uL. Transferring every 100 uL into a 1.5 mL centrifuge tube and freezing at −80° C. in a refrigerator.

Introduction of auxiliary plasmid pKD46: taking a strain of E. coli MG1655 from a −80° C. refrigerator, transferring it to the competent state, thawing it on ice, mixing it thoroughly with about 50 ng of plasmid pKD46, transferring it to a precooled 1 mm electroporation cuvette, and icing bath for 10 minutes. Wiping off the water mist on the outside of the electroporation cuvette with paper, placing it in an electric rotation instrument, and using Ecl level to shock once. Immediately adding 600 uL of precooled SOC culture medium (Super Optimal Broth with Catabolite repression), gently mixing well, and tilting the electroporation cuvette; all the mixture is transferred to a sterile 1.5 mL centrifuge tube. At 30° C., 200 rpm, shaking for 80 min, taking 80 uL and coating it onto the LB plates containing ampicillin sodium. Incubating overnight at 30° C.

(3) Insertion of Linearized Fragment KO-Δ pykF

Positive clones from step (2) are selected and they are inoculated into a test tube containing 5 mL of LB and ampicillin sodium at 30° C., 200 rpm, and shaking for 16-20 hours. Inoculating 2% volume into 15 mL of fresh LB medium at 30° C., 200 rpm, and shaking for 2 hours, with OD600=0.4-0.6. Centrifuging and discarding the supernatant, washing twice with 10% glycerol, and finally adding a certain amount of 10% glycerol to make a final volume to be 100 uL. Transferring to a 1.5 mL centrifuge tube and mixing thoroughly with approximately 500 ng of fragment KO-Δ pykF. Performing electroconvulsive transformation using the same method as step (2), and applying the recovered mixture to LB plates containing ampicillin sodium and chloramphenicol. Incubating overnight at 30° C. Using Seq-Δ pykF_F and Seq-KO_R as primers, colony PCR was performed on the positive clones on the plate to screen for positive clones.

(4) Elimination of Auxiliary Plasmid pKD46

Drawing the positive clones from step (3) onto the LB plates containing chloramphenicol and incubating overnight at 42° C. Transferring the positive clones on the plate into 5 mL LB tubes containing or not containing ampicillin sodium to verify the elimination of plasmid pKD46.

### (5) Elimination of Screening Marker CmR

Inoculating the strain with plasmid pKD46 eliminated into a test tube containing 5 mL LB and chloramphenicol at 37° C., 200 rpm, and shaking for 12-16 hours. Inoculating 2% volume into 15 mL fresh LB medium, 37° C., 200 rpm, shaking for 1.5 hours, OD600=0.4-0.6, centrifuging and discarding the supernatant, washing twice with 10% glycerol, and finally adding a certain amount of 10% glycerol to make the final volume to be 100 uL. Transferring to a 1.5 mL centrifuge tube and mixing thoroughly with about 50 ng plasmid pjw168. Performing electroporation transformation using the same method as step (2), and coating it onto the LB plates containing ampicillin sodium and 1 mM IPTG. Incubating overnight at 30° C. Using Seq-Δ pykF_F and Seq-Δ pykF_R as primers, colony PCR was performed on the positive clones on the plate to screen for positive clones.

### (6) Elimination of Auxiliary Plasmid Pjw168

Drawing the positive clones from step (5) onto LB plates and incubating overnight at 42° C. Transferring the positive clones on the plate into 5 mL LB tubes containing or not containing ampicillin sodium to verify the elimination of plasmid pjw168. The successfully eliminated strain is strain MG1655ΔpykF.

TABLE 1
Genotypes of strains and plasmids

| Strain/plasmid | Genotype |
|---|---|
| Strain | |
| DH5α | F- endAl gln V44 thi-1 recA1 relA1 gyrA96 deoR nupG purB20 φ80dlacZΔM15 |
| | Δ(lacZYA-argF)U169, hsdR17(r_{K}⁻m_{K}⁺), λ⁻ |
| MG1655 | F⁻ λ⁻ ilvG- rfb-50 rph-1 |
| LPAA01 | MG1655 harboring pTrc99A |
| LPAA02 | MG1655 harboring pLPAA02 |
| LPAA03 | MG1655 harboring pLPAA03 |
| LPAA04 | MG1655 harboring pLPAA04 |
| LPAA05 | MG1655 harboring pLPAA02 and pLPAA05 |
| LPAA06 | MG1655ΔpykF harboring pLPAA02, pLPAA05 and pLPAA06 |
| Plasmid | |
| pTrc99A | Ap^{R}, trc promoter, pBR322 origin, lacI^{q} |
| pBBR1Tac | Cm^{R}, tac promoter, pBBR1 origin |
| pCDFTac | Sm^{R}, tac promoter, CDF origin, lacI |
| pLPAA01 | pTrc99A derivative containing E. coli-codon optimized g12203^{opt} encoding |
| | O-methyltransferase (PSA-OMT1) from Pleurotus sapidus |
| pLPAA02 | pLPAA01 derivative containing RBS-ubiC from MG1655 |
| pLPAA03 | pLPAA01 derivative containing RBS-PrubiC^{opt} from Providencia rustigianii |
| pLPAA04 | pLPAA02 derivative containing tac-RBS-aroG^{D146N} from MG1655- rrnBT1T2 cassette |
| pLPAA05 | pBBR1Tac derivative containing aroG^{D146N} from MG1655 |
| pLPAA06 | pCDFTac derivative containing ppsA from MG1655 |
| pKD46 | Ap^{R}, λ-Red recombinase under arabinose-inducible araBAD promoter, ts origin |
| pJW168 | Ap^{R}, Cre-recombinase under IPTG-inducible lacUV5 promoter, ts origin |
| pUTrc | Ap^{R}, Cm^{R}, modified pU100 containing trc promoter downstream of lox66-cat-lox71 |
| | cassette |

TABLE 2
Sequence listing of Primers

| Primer | Sequence listing (5′→3′) |
|---|---|
| PSA-OMT1_F | TCACACAGGAAACAGACCATGACCACCCTGAGCC |
| | CG |
| PSA-OMT1_R | CGGGTACCGAGCTCGAATTCTTACGCCGGAACAA |
| | ATTCGGTAACG |
| Seq-pTrc99A_F | CGCCGACATCATAACGGTTC |
| Seq-pTrc99A_R | TACTGCCGCCAGGCAAATTC |
| ubiC_F | TCGGTACCCGGGGATCCTTTAATAAGGAGATATA |
| | CCATGTCACACCCCGCGTTAAC |
| ubiC_R | CATGCCTGCAGGTCGACTTTAGTACAACGGTGAC |
| | GCCG |
| PrubiC_F | TCGGTACCCGGGGATCCTTTAATAAGGAGATATA |
| | CCATGCATGAAACAATATTTACTC |
| PrubiC_R | CATGCCTGCAGGTCGACTTTAACGATATGCCGGA |
| | G |
| pBBR1Tac_F | GGATCCTCTAGAGTCGACC |
| pBBR1Tac_R | GAATTCTGTTTCCTGTGTGAAATTG |
| aroG1_F | CACACAGGAAACAGAATTCATGAATTATCAGAAC |
| | GACGATTTACGC |
| aroG1_R | GGGGTGATCATATTGAGAAACTCACCTGC |
| aroG2_F | TTTCTCAATATGATCACCCCACAATATCTCGC |
| aroG2_R | GTCGACTCTAGAGGATCCTTACCCGCGACGCGCT |
| | TTTAC |
| Seq-pBBR1Tac_F | CTATTTAACGACCCTGCCC |
| ubiCT_F | TAAGAATTCGAGCTCGGTACCCGG |
| ubiCT_R | GAATATTTGCCAGAAACGCAAAAAGGCCATCCGT |
| | C |
| tac-aroG_F | TGCGTTTCTGGCAAATATTCTGAAATGAGCTGTT |
| | GAC |
| tac-aroG_R | CATCCGCCAAAACAGCCAAG |
| ppsA_F | CAATTTCACACAGGAAACAGAATTCATGTCCAAC |
| | AATGGCTCGTC |
| ppsA_R | ATCCCCGGGTACCGAGCTCTTATTTCTTCAGTTC |
| | AGCCAGGC |
| Seq-pCDFTac_F | AATTGACTCTCTTCCGGGCG |
| ΔpykF-up_F | AAATCAAACAAAATCAGACAAATAAC |
| ΔpykF-up_R | CGCGTTCTATAGTGTCACCATGACAGTCTTAGTC |
| | TTTAAG |
| ΔpykF-down_F | CCCATCAGATCCACTAGTGCATCTGTTCACGTCC |
| ΔpykF-down_R | CCTGAACGTCGGAACG |
| KO_F | GTGACACTATAGAACGCG |
| KO_R | ACTAGTGGATCTGATGGG |
| Seq-ApykF_F | TTAGAGCGAGGCACCACC |
| Seq-KO_R | ATACCACGACGATTTCCGGC |
| Seq-ApykF_R | GGATGCTTCCATCGGATTCATC |

### Example 4: Construction of LPAA01~LPAA06 Strains

Inoculating E. coli MG1655 into a test tube containing 5 mL LB, at 37° C., 200 rpm, and shaking for 12-16 hours. Inoculating 2% volume into 100 mL fresh LB medium, at 37° C., 200 rpm, and shaking for 1.5 hours, OD600=0.4-0.6. Centrifuging and discarding the supernatant, washing twice with 10% glycerol, and finally adding a certain amount of 10% glycerol to make the final volume 500 uL. Dividing every 100 uL into a 1.5 mL centrifuge tube. Mixing four of the electropositive states with approximately 50 ng plasmids pTrc99A, pLPAA02, pLPAA03, and pLPAA04, respectively. Performing electroporation in the same manner as in step (2) of Example 3, and coating them onto the LB plates containing ampicillin sodium. Incubating overnight at 37° C. to obtain LPAA01, LPAA02, LPAA03, and LPAA04, respectively. Mixing one competent vector with 50 ng of plasmids pLPAA02 and pLPAA05, and performing electroporation in the same manner as in step (2) of Example 3. Coating it onto the LB plates containing ampicillin sodium and chloramphenicol, and culturing overnight at 37° C. to obtain LPAA05.

Preparing the competent state of MG1655ΔpykF according to the above method, and sequentially introduce plasmids pLPAA02, pLPAA05, and pLPAA06. Paying attention to adding corresponding resistance to each culture medium, and finally obtaining the strain LPAA06.

### Example 5: Fermentation of LPAA01~LPAA05 Strains

Seed cultivation: selecting the positive clone from the corresponding plate and inoculating it into a test tube containing 5 mL of seed culture medium (LB medium). Incubating overnight at 37° C. and 200 rpm.

Shake flask fermentation: inoculating the seed solution at a 2% inoculation rate into a 250 mL shaker flask containing 50 mL of fermentation medium. Shaking the flask at 30° C. and 200 rpm for 60 hours. After 8 hours of inoculation (OD600=0.5~0.8), adding IPTG at a final concentration of 1 mM. Performing three biological replicates per group.

The composition of shake flask fermentation medium is glucose 10 g/L, MgSO₄ 7H₂O 0.8 g/L, KH₂PO₄ 6.67 g/L, (NH₄) ₂HPO₄ 4 g/L, citric acid 0.8 g/L, trace metal salt solution 5 mL/L, pH 7.0. The stock concentrations of glucose and MgSO₄·7H₂O were 500 g/L and 80 g/L, respectively, and subjected to independent wet heat sterilization. Composition of trace metal salt solution: 10 g/L FeSO₄·7H₂O, 2.65 g/L CaCl₂·2H₂O, 2.2 g/L ZnSO₄·7H₂O, 0.58 g/L MnSO₄·5H₂O, 1 g/L CuSO₄·5H₂O, 0.1 g/L (NH₄)₆Mo₇O₂₄·4H₂O, 0.02 g/L Na₂B₄O₇·10H₂O, 10 mL/L 35% HCl, the solvent is deionized water.

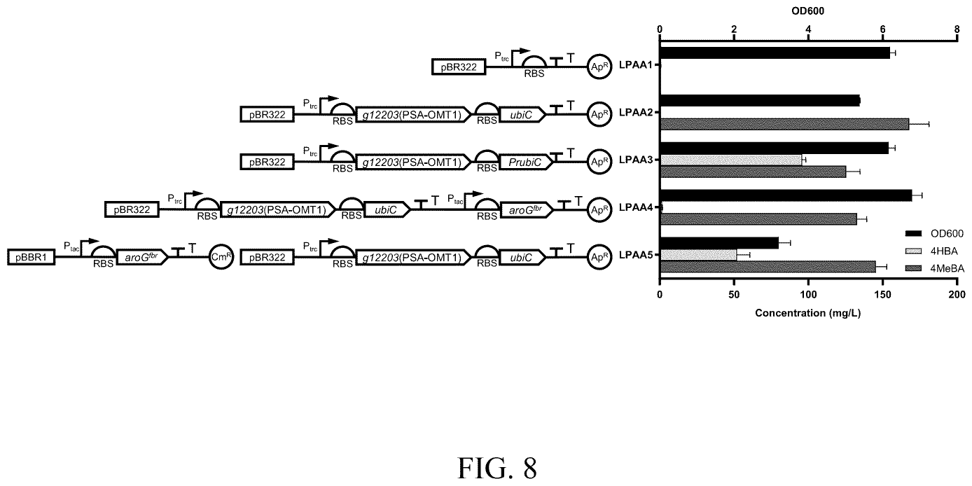

After fermentation, taking 1 mL of fermentation broth, of which 100 uL is used to dilute 10 times to detect the OD value of the bacterial solution, and the remaining 900 uL is centrifuged at 16000×g for 10 minutes. Taking the supernatant and filtering it through a 0.22 μm aqueous phase filter membrane, diluting it 5 times, and detecting it by high performance liquid chromatography. The results are shown in FIG. 8, and the yield of p-anisic acid in LPAA02 is 167.9 mg/L. The yield of p-anisic acid in LPAA05 reached 145.4 mg/L, with 52.0 mg/L of p-hydroxybenzoic acid remaining, and a low OD600 value, which may be due to the high concentration of the inducer IPTG. Meanwhile, insufficient supply of the cofactor SAM may also be a factor limiting the production of p-anisic acid.

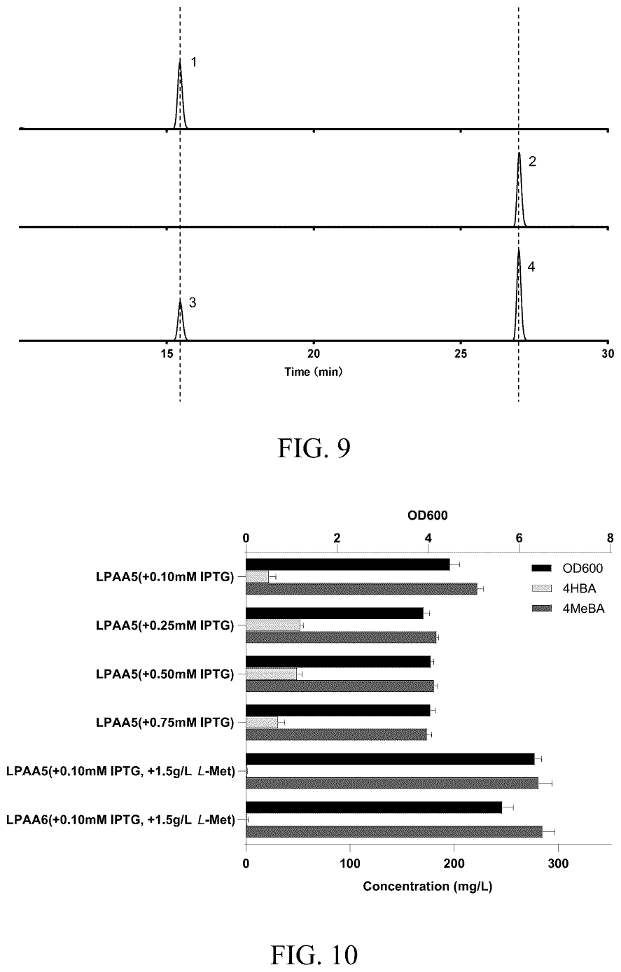

The high-performance liquid chromatography system is Shimadzu LC-20 series, and the chromatographic column is Agilent Poroshell 120 EC-C18 (4.6× 150 mm). Phase A is ddH₂O containing 0.1% trifluoroacetic acid, phase B is methanol, flowed at 0.65 mL/min according to the following procedure: 0-1 min, 10% phase B; 1-21 minutes, phase B increases linearly from 10% to 50%; 21-22 minutes, phase B increases linearly from 50% to 70%; 22-24 min, 70% phase B; 24-26 minutes, phase B decreases linearly from 70% to 10%; 26-35 min, 10% phase B. The injection volume is 5 uL, the column temperature is 30° C., and the detection wavelength of the UV visible spectrophotometer is 246 nm. As shown in FIG. 9, peak 1 is a standard p-hydroxybenzoic acid, and a peak time is 15.225 min; peak 2 is a standard substance of p-anisic acid, with a peak time of 26.738 minutes. Peak 3 and Peak 4 are samples of LPAA3, identified as p-hydroxybenzoic acid and p-anisic acid, respectively.

### Example 6: Fermentation of LPAA05 and LPAA06 Under Different Conditions

LPAA05 was subjected to shake flask fermentation in the same manner as Example 5, but after 8 hours of inoculation (OD600=0.5~0.8), IPTG with final concentrations of 0.10 mM, 0.25 mM, 0.50 mM, and 0.75 mM were added. The same method as in Example 5 was used for detection, and the results are shown in FIG. 10. When the IPTG concentration was 0.1 mM, the yield of p-anisic acid reached 222.3 mg/L, with 22.2 mg/L of p-hydroxybenzoic acid remaining.

Using the same method as in Example 5, LPAA05 and LPAA06 were subjected to shake flask fermentation, but after 8 hours of inoculation (OD600-0.5~0.8), IPTG with a final concentration of 0.10 mM and 1.5 g/L L-methionine were added. The L-methionine stock concentration was 50 g/L, and the bacteria were independently filtered and sterilized. The same method as in Example 5 was used for detection, and the results are shown in FIG. 9. Under these conditions, LPAA05 produced 281.0 mg/L of p-anisic acid, and LPAA06 produced 284.9 mg/L of p-anisic acid, with almost no residual hydroxybenzoic acid. Specifically, after adding L-methionine, LPAA05 showed a significant increase in OD600, indicating that insufficient SAM supply also affected the growth of the strain.

After genetic engineering, the genetically engineered bacterium LPAA06 produced 284.9 mg/L of p-anisic acid through shake flask fermentation. Therefore, the genetically engineered strain of p-anisic acid constructed by the present disclosure can achieve effective accumulation of p-anisic acid in the fermentation broth during the fermentation process, laying the foundation for the construction of high-yield p-anisic acid genetically engineered strains.

The above are only preferred embodiments of the present disclosure and are not intended to limit the present disclosure. For those skilled in the art, the present disclosure may have various modifications and variations. Any modifications, equivalent substitutions, improvements, etc. made within the spirit and principles of the present disclosure shall be included within the protection scope of the present disclosure.

What is claimed is:

1. A recombinant Escherichia coli strain for de novo biosynthesis of p-anisic acid, wherein at least one of the following improvements has been made to a starting strain of E. coli:
(1) overexpression of O-methyltransferase PSA-OMT1 from Pleurotus sapidus; overexpression of UbiC, a chorismic acid lyase derived from E. coli MG1655 or Providencia rustigianii;
(2) on the basis of (1), overexpression of a feedback inhibition mutant AroG^{D146N} derived from 3-deoxy-D-arabino-heptulosonate 7-phosphate, (DAHP) synthase of E. coli MG1655;
(3) on the basis of (2), overexpression of PpsA derived from phosphoenolpyruvate, (PEP) synthase, and knockout of pykF gene encoding a pyruvate kinase I;
a nucleotide sequence of codon optimized O-methyltransferase PSA-OMT1 is shown in SEQ ID No.: 1;
a nucleotide sequence of the chorismic acid lyase UbiC derived from E. coli MG1655 is shown in SEQ ID No.: 2;
a nucleotide sequence of codon optimized chorismic acid lyase UbiC derived from Providencia rustigianii is shown in SEQ ID No.: 3;
a nucleotide sequence of the feedback inhibition mutant AroG^{D146N} of DAHP synthase is shown in SEQ ID No.: 4;
a nucleotide sequence of the PEP synthase PpsA is shown in SEQ ID No.: 5.

2. The recombinant Escherichia coli according to claim 1, wherein the O-methyltransferase PSA-OMT1 and the chorismic acid lyase UbiC are expressed using pTrc99A as an expression vector.

3. The recombinant Escherichia coli according to claim 1, wherein the feedback inhibition mutant AroG^{D146N} of (DAHP synthase is expressed using pTrc99A or pBBR1Tac as an expression vector.

4. The recombinant Escherichia coli according to claim 1, wherein the PEP synthase PpsA is expressed using pCDFTac as an expression vector.

5. The recombinant Escherichia coli according to claim 1, wherein the recombinant E. coli is derived from E. coli MG1655 as a starting strain.

6. A method for producing p-anisic acid, wherein the method comprises using recombinant E. coli as a fermentation strain according to claim 1, and glucose is fermented as a substrate to produce the p-anisic acid.

7. The method for producing p-anisic acid according to claim 6, wherein the recombinant E. coli is inoculated into a fermentation medium and cultured at 28-32° C. and 100-500 rpm for 48-72 hours; when OD600=0.5-0.8, isopropyl-beta-D-thiogalactopyranoside, (IPTG) with a final concentration of 0.1-1.0 mM is added for induction, after fermentation, a supernatant is separated and purified to obtain the p-anisic acid.

8. The method for producing p-anisic acid according to claim 7, wherein the fermentation medium comprise glucose, MgSO₄·7H₂O, KH₂PO₄, (NH₄)₂HPO₄, Citric acid, L-methionine, trace metal salt solution; and deionized water, and a pH value of the fermentation medium is 6.5~7.5.

9. An application of the method for producing p-anisic acid according to claim 6 in the preparation of products containing p-anisic acid by microbial fermentation.
